(19) United States
(12) Patent Application Publication
(10) Pub. No.: US 2024/0318259 A1
(43) Pub. Date: Sep. 26, 2024

(54) METHOD FOR DETERMINING PRIMARY TUMOR SITE

(71) Applicant: ONCOCROSS CO., LTD., Seoul (KR)

(72) Inventors: Young Heun LEE, Seoul (KR); Yi Rang KIM, Sejong (KR); Ji Hoon KANG, Seoul (KR)

(21) Appl. No.: 18/278,664

(22) PCT Filed: Sep. 23, 2022
(86) PCT No.: PCT/KR2022/014283

§ 371 Date: Aug. 24, 2023

(30) Foreign Application Priority Data
Sep. 24, 2021 (KR) 10-2021-0126397
Sep. 23, 2022 (KR) 10-2022-0120857

(51) Int. Cl. C12Q 1/6886

(52) U.S. Cl. CPC C12Q 1/6886; C12Q 2600/158

(57) ABSTRACT

Disclosed is a method for diagnosing carcinoma of unknown primary, using artificial intelligence. A diagnostic method for carcinoma of unknown primary, using artificial intelligence according to an embodiment of the present invention includes the steps of: producing gene expression pattern information of a sample collected from a tissue where metastatic cancer is generated; removing already learned gene expression pattern information attributed to the tissue from the gene expression pattern information of the sample collected from the tissue where metastatic cancer is generated; comparing the gene expression pattern information deprived of the tissue-attributed gene expression pattern information with gene expression pattern information by carcinoma; and specifying a primary site of the sample collected from the tissue where the metastatic cancer is generated.

### BACKGROUND

### Field

The present invention relates to a method for determining a primary tumor site, and more particularly, to a method for determining the primary tumor site using a gene expression pattern of a biological specimen including tumor cells.

### Related Art

Cells, the smallest unit of the body, have their own order and self-regulating function to keep their number in balance. However, when the number of newly created cells exceeds that of dying cells with unknown cause, unnecessary extra cells do not perform their role properly and clump together in one place to settle down.

This form is called a tumor. The tumor in a state in which the tumor does not stop at a certain size and constantly proliferates and invades surrounding normal cells is defined as a malignant tumor, that is, cancer.

Cancer may be divided into primary cancer, in which cancer cell tissues first settle down and begin to be formed, and metastatic cancer, which is generated in other organs by moving cancer cells from the primary organ along blood vessels or lymphatic vessels.

Since metastasis cancer shares biochemical characteristics with primary cancer, treatment methods that are similar to those applied to primary cancer are applied to metastatic cancer regardless of the location where the metastatic cancer is generated. Accordingly, in selecting the optimal therapeutic agent or treatment method, the stage of specifying the primary site of cancer needs to be preceded.

For most metastatic cancers, the primary site may be specified through pathological examination of a sample, but in some cases, the primary site may not be specified even after immunohistochemical staining, molecular genetic testing, and tumor marker testing are performed. This is called Carcinoma of Unknown Primary (CUP).

Until now, a combination treatment with multiple alkaloid-based anti-malignant-tumor agents (for example, paclitaxel, carboplatin, etc.) is known as the standard treatment for patients with cancer of unknown primary site. Nevertheless, it has been reported that the 5-year average survival rate is significantly lower than that of other cancers.

Accordingly, the need for a new type of primary site determination method capable of specifying the primary site of cancer of unknown primary site has emerged.

### SUMMARY

The present invention has been devised to obviate the above limitation. An aspect of the present invention is directed to providing a method for specifying a primary site of cancer using gene expression pattern information of a biological specimen including tumor cells.

The aspect of the present invention is not limited to those mentioned above, and other aspects not mentioned herein will be clearly understood by those skilled in the art from the following description.

A method for determining a primary tumor site according to an embodiment of the present invention includes: acquiring gene expression data of a biological sample including tumor cells of which a primary site is not specified; and classifying the primary site of the biological sample into one of a plurality of tumor types by comparing the gene expression data of the biological sample with specific gene expression data for each of the plurality of tumor types using a classification algorithm.

According to the aforementioned method for diagnosing cancer of unknown primary site, in specifying the primary site of cancer of unknown primary site using a gene expression pattern, it is possible to exclude gene expression patterns attributed to the tissues where metastatic cancer is generated, thus further improving the accuracy of diagnosis.

### DESCRIPTION OF EXEMPLARY EMBODIMENTS

Hereinafter, preferred embodiments of the present disclosure will be described in detail with reference to the accompanying drawings. The advantages and features of the present disclosure and methods of achieving the same will be apparent from the embodiments that will be described in detail with reference to the accompanying drawings. It should be noted, however, that the technical ideas of the present disclosure are not limited to the following embodiments, and may be implemented in various different forms. Rather the embodiments are provided so that the technical ideas of the present disclosure will be thorough and complete and will fully convey the scope of the present disclosure to those skilled in the technical field to which the present disclosure pertains. It is to be noted that the technical ideas of the present disclosure are defined only by the claims.

In adding reference numerals for elements in each drawing, it should be noted that like reference numerals designate like elements wherever possible even though elements are shown in other drawings. Furthermore, in describing the present disclosure, a detailed description of the related known functions and constructions will be omitted if it is deemed to make the gist of the present disclosure vague.

Unless otherwise defined, all terms including technical and scientific terms used herein have the same meaning as commonly understood by those skilled in the technical field to which the present disclosure pertains. It will be understood that terms, such as those defined in commonly used dictionaries, should be interpreted as having a meaning that is consistent with their meaning in the context of the relevant art and will not be interpreted in an idealized or overly formal sense unless expressly so defined herein. Terms used in the specification are used to describe embodiments of the present disclosure and are not intended to limit the scope of the present disclosure. In the specification, the terms in singular form may include plural forms unless otherwise specified.

In addition, in the description of the components of the embodiment of the present disclosure, the terms such as first, second, A, B, (a), and (b) may be used. These terms are merely used to distinguish the components from other components, and do not delimit an essence, an order or a sequence of the corresponding components. When it is described that a component is “connected”, “coupled”, or “jointed” to another component, the description may include not only being directly connected, coupled or joined to the other component but also being “connected” “coupled” or “joined” by another component between the component and the other component.

The terms “comprises” and/or “comprising” used herein do not preclude the presence or addition of one or more other components, steps, operations, and/or elements, in addition to the mentioned components, steps, operations, and/or elements.

### Informative-Genes

The expression levels of genes of the present invention have been identified as providing useful information regarding the primary site of tumor cells. These genes are referred to herein as “informative-genes.” Informative-genes include protein coding genes and non-protein coding genes. The expression levels of informative-genes may be measured by evaluating the levels of appropriate gene products (for example, mRNAs, miRNAs, proteins etc.).

Table 3 below provides a listing of specific informative-genes that are differentially expressed for each primary site of tumor cells.

Certain methods described herein includes measuring expression levels in the biological sample of at least one informative-gene. However, in some embodiments, the expression analysis involves measuring the expression levels in the biological sample of at least 2, at least 3, at least 4, at least 5, at least 6, at least 7, at least 8, at least 9, at least 10, at least 20, at least 30, at least 40, at least 50, at least 60, at least 70 or at least 80 informative-genes. In some embodiments, as shown in Table 11, the expression analysis involves measuring expression levels in the biological sample of 1 to 5, 1 to 10, 5 to 10, 5 to 15, 10 to 15, 10 to 20, 15 to 20, 15 to 25, 20 to 30, 25 to 50, 25 to 75, 50 to 100, 50 to 200 or more informative-genes. In some embodiments, as shown in Table 11, the expression analysis involves measuring expression levels in the biological samples of at least 1 to 5, 1 to 10, 2 to 10, 5 to 10, 5 to 15, 10 to 15, 10 to 20, 15 to 20, 15 to 25, 20 to 30, 25 to 50, 25 to 75, 50 to 100, 50 to 200 or more informative-genes.

In some embodiments, the number of informative-genes for an expression analysis are sufficient to provide a level of confidence in a prediction outcome that is clinically useful. This level of confidence (for example, strength of a prediction model) may be assessed by a variety of performance parameters including, but not limited to, the accuracy, sensitivity specificity, and area under the curve (AUC) of the receiver operator characteristic (ROC). These parameters may be assessed with varying numbers of features (for example, number of genes, mRNAs) to determine an optimum number and set of informative-genes. An accuracy, sensitivity or specificity of at least 60%, 70%, 80%, 90%, may be useful when used alone or in combination with other information.

Any appropriate system or method may be used for determining expression levels of informative-genes. Gene expression levels may be measured through the use of a hybridization-based assay. As used herein, the term “hybridization-based assay” refers to any assay that involves nucleic acid hybridization. A hybridization-based assay may or may not involve amplification of nucleic acids.

Hybridization-based assays are well known in the art and include, but are not limited to, array-based assays (for example, oligonucleotide arrays, microarrays), oligonucleotide conjugated bead assays (for example, Multiplex Bead-based Luminex® Assays), molecular inversion probe assays, and quantitative RT-PCR assays. Multiplex systems, such as oligonucleotide arrays or bead-based nucleic acid assay systems are particularly useful for evaluating levels of a plurality of genes simultaneously. Other appropriate methods for measuring levels of nucleic acids will be apparent to those skilled in the art.

As used herein, a “level” refers to a value indicative of the amount or occurrence of a substance, for example, an mRNA. A level may be an absolute value, for example, a quantity of mRNA in a sample, or a relative value, for example, a quantity of mRNA in a sample relative to the quantity of the mRNA in a reference sample (control sample). The level may also be a binary value indicating the presence or absence of a substance. For example, a substance may be identified as being present in a sample when a measurement of the quantity of the substance in the sample, for example, a fluorescence measurement from a PCR reaction or microarray, exceeds a background value. Similarly, a substance may be identified as being absent from a sample (or undetectable in the sample) when a measurement of the quantity of the molecule in the sample is at or below background value.

It should be appreciated that the level of a substance may be measured directly or indirectly.

### Biological Samples

The method for determining the primary tumor site according to an embodiment of the present invention begins with acquiring a “biological sample.” As used herein, the phrase “acquiring a biological sample” refers to any process for directly or indirectly acquiring a biological sample from a subject.

In an embodiment, the term “biological sample” refers to a specimen of biological tissue or biological fluid including nucleic acids. Such specimens include, but are not limited to, tissue or fluid isolated from a subject. Biological specimens may also include sections of tissues such as biopsy and autopsy specimens, FFPE specimens, frozen sections taken for histological purposes, blood, plasma, serum, sputum, stool, tears, mucus, hair, and skin. Biological specimens also include explants and primary and/or transformed cell cultures derived from animal or patient tissues.

Biological specimens may also be blood, a blood fraction, urine, effusions, ascitic fluid, saliva, cerebrospinal fluid, cervical secretions, vaginal secretions, endometrial secretions, gastrointestinal secretions, bronchial secretions, sputum, cell line, tissue specimen, cellular content of fine needle aspiration (FNA) or secretions from the breast.

A biological specimen may be provided by removing a specimen of cells from an animal, but may also be provided using previously isolated cells or by performing the methods described herein in vivo.

A biological sample may be processed in any appropriate manner to facilitate determining expression levels. For example, biochemical, mechanical and/or thermal processing methods may be appropriately used to isolate a biomolecule of interest, for example, RNA, from a biological sample. Accordingly, a RNA or other molecules may be isolated from a biological sample by processing the sample using methods well known in the art.

### Determination of Informative Gene Expression

The method for determining the primary tumor site according to an embodiment of the present invention may include comparing an informative gene expression level of a biological sample including tumor cells with one or more reference values.

The term “reference value” refers to the expression level (or expression level range) of informative genes specifically expressed for each primary site. For example, an appropriate criterion may represent the expression level of an informative gene in a reference (control) biological sample obtained from a subject of known primary site.

For example, in the case where the informative gene specifically expressed in a biological sample whose primary site is Adenoid Cystic Carcinoma (ACC) is specified as CBLN4, FMO2, PTH1R, or TH, when the expression levels of CBLN4, FMO2, PTH1R, and TH in the biological sample collected from a test target are all above the reference value or exceed the reference value, the tumor to be tested may be specified as ACC, considering that all informative genes related to ACC are expressed.

The determination of whether the expression level of the informative gene of the biological sample collected from a test subject has reached a “reference value” may be determined in various ways. For example, the “reference value” may be determined to be reached when the expression level of a particular gene in a biological sample is at least 1%, at least 5%, at least 10%, at least 25%, at least 50%, at least 100%, at least 250%, at least 500%, or at least 1,000% higher or lower than the reference value of that gene.

Similarly, when the expression level of the informative gene in a biological sample is at least 1.1-fold, 1.2-fold, 1.5-fold, 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher, or lower, than the reference value of that gene, the gene may be determined to be expressed above the “reference value.”

However, the determination of whether a specific gene included in the biological sample is expressed above a reference value may be made in various ways.

### Primary Site Determination Model of Tumor Cells Included in Biological Samples

The method for determining the primary tumor site according to an embodiment of the present invention includes: comparing a set of expression levels (which may also be referred to as an expression pattern or profile) of an informative gene in a biological sample obtained from a test subject with a plurality of sets of reference levels (which may also be referred to as a reference pattern); identifying a reference pattern most similar to the expression pattern; and classifying the biological sample of a test target into one of a plurality of tumor types by matching the reference pattern with the expression pattern of a tumor whose primary site is specified.

The method may involve constructing or configuring a predictive model, which may be referred to as a classifier or predictor, that may be used to classify a primary site of a biological sample including tumor cells into at least one of a plurality of tumor types.

The term “primary tumor site classifier” used herein is a model that probabilistically predicts the primary site of a subject based on the expression level measured in a biological sample obtained from a test subject. Typically, models are constructed using specimens for which the classification (tumor with a specified primary site) has presently been identified. Once the model (classifier) is constructed, expression levels obtained from a biological sample of a test subject whose primary site is unknown may be applied to predict the primary site of tumors in the biological sample of the subject.

The classification method may involve classifying a primary site of tumor cells included in a biological sample into at least one type among a plurality of tumor types, and calculating a probability that the tumor cells correspond to a specific tumor type. For example, it is possible to calculate the probability that the tumor cells included in the biological sample are ACC (Adenoid Cystic Carcinoma), ATC (Anaplastic Thyroid Carcinoma), BCC (Basal Cell Carcinoma), and the like. The method for determining the primary tumor site according to an embodiment of the present invention may output result values for each tumor type with a high probability, or may specify and output a tumor type with a probability greater than or equal to a predetermined threshold value as a primary site.

It should be understood that various predictive models known in the art may be used as primary tumor site classifiers. For example, the primary tumor site classifier may include an algorithm selected from logistic regression, partial least squares, linear discriminant analysis, quadratic discriminant analysis, neural network, naïve Bayes, C4.5 decision tree, k-nearest neighbor, random forest, support vector machine, or other appropriate method.

The primary tumor site classifier may be trained on a data set including expression levels of the plurality of informative-genes in biological samples with specified primary site. For example, the primary tumor site classifier may be trained on a data set including expression levels of a plurality of informative-genes in biological samples obtained from a plurality of subjects with specified primary site based histological findings.

Once a model is constructed, the validity of the model may be tested using methods known in the art. One way to test the validity of the model is by cross-validation of the dataset. To perform cross-validation, one, or a subset, of the samples is eliminated and the model is constructed, as described above, without the eliminated sample, forming a “cross-validation model.” The eliminated sample is then classified according to the model, as described herein. This process is completed with all the samples, or subsets, of the initial dataset and an error rate is measured. The accuracy the model is then assessed. This model classifies samples to be tested with high accuracy for classes that are known, or classes have been presently identified. Another way to validate the model is to apply the model to an independent data set, such as a new biological sample including tumor cells of which a primary site is not specified.

### Implementation of Model for Determining Primary Site of Tumor Cells Included in Biological Sample Using Computing Device

Methods described herein may be implemented in any of numerous ways. For example, certain embodiments may be implemented using hardware, software or a combination thereof. When implemented in software, the software code may be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other portable or fixed electronic device.

In addition, a computer may have one or more input and output devices. These devices may be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

In addition, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the aspects of the present invention may be embodied as a computer readable medium (or multiple computer readable media) (for example, a computer memory, one or more floppy discs, compact discs (CD), optical discs, digital video disks (DVD), magnetic tapes, flash memories, circuit configurations in Field Programmable Gate Arrays or other semiconductor devices, or other non-transitory, tangible computer storage medium) encoded with one or more programs that, when executed on one or more computers or other computers, perform methods that implement various embodiments of the present invention discussed above. The computer readable medium or media may be transportable, such that the program or programs stored thereon may be loaded onto one or more different computers or other processors to implement various aspects of the present invention as discussed above. As used herein, the term “non-transitory computer-readable storage medium” encompasses only a computer-readable medium that may be considered to be a manufacture (in other words, article of manufacture) or a machine.

The terms “program” or “software” are used herein in a generic sense to refer to any type of computer code or set of computer-executable instructions that may be employed to program a computer or other processors to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

As used herein, the term “database” generally refers to a collection of data arranged for ease and speed of search and retrieval. Further, a database typically includes logical and physical data structures. Those skilled in the art will recognize the methods described herein may be used with any type of database including a relational database, an object-relational database and an XML-based database, where XML stands for “extensible-MarkupLanguage.” For example, the gene expression information may be stored in and retrieved from a database. The gene expression information may be stored in or indexed in a manner that relates the gene expression information with a variety of other relevant information (for example, information relevant for creating a report or document that aids in establishing treatment protocols and/or making diagnostic determinations, or information that aids in tracking patient samples). Such relevant information may include, for example, patient identification information, ordering physician identification information, information regarding an ordering physician's office (for example, address, telephone number), information regarding the origin of a biological sample (for example, tissue type, date of sampling), biological sample processing information, specimen quality control information, biological sample storage information, gene annotation information, etc.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

In some aspects of the present invention, computer implemented methods for processing genomic information are provided. The methods involve: acquiring gene expression data of a biological sample including tumor cells of which a primary site is not specified; and classifying the primary site of the biological sample into one of a plurality of tumor types by comparing the gene expression data of the biological sample with specific gene expression data for each of the plurality of tumor types using a classification algorithm. Any of the statistical or classification methods described herein may be incorporated into the computer implemented methods. In some embodiments, the methods involve calculating a probability that the tumor cells included in the biological sample are of at least one of a plurality of tumor types of which the primary site is specified. The computer implemented methods may involve generating a report indicating the probability that tumor cells included in the biological sample are of the tumor type of which the primary site is specified. Such methods may also involve transmitting the report to a health care provider of a subject.

### Example 1. Collection of Gene Expression Data for Plurality of Tumor Types with Specified Primary Sites

The gene expression data and clinical information for a plurality of tumor types with specified primary sites was obtained from GEO (Gene Expression Omnibus, https://www.ncbi.nlm.nih.gov/geo/, Applicable platforms: GPL570, A-AFFY-44), a public database, ArrayExpress, TCGA, ICGS, and GTEx.

### Expression Data

illumina TrueSeq RNA sequencing
Affymetrix Human Gene 1.1 ST Expression Array (V3; 837 samples)

### Genotype Data

Whole genome sequencing (HiSeq X; first batch on HiSeq 2000)
Whole exome sequencing (Agilent or ICE target capture, HiSeq 2000)
Illumina OMNI 5M Array or 2.5M SNP Array
Illumina Human Exome SNP Array

### Analysis Methods

Updated on Aug. 20, 2019
Current Release: V8

### General Sample Collection

Genome Tissue Expression (GTEx) SOPs
Current Release: V8

Among the gene expression data obtained from the database, gene expression data of 20,267 cancer patients and gene expression data of 12,490 normal tissues were used for model development.

After filtering the collected data (filtering conditions: Homo sapiens, Tissue Biopsy), various tumor types included in the data were classified into 42 types. Tumors classified as the same type are tumors with clinically similar characteristics. The 42 tumor types are listed in the table below.

TABLE 1

| Order | Cancer Type | DESCRIPTION |
|---|---|---|
| 1 | ACC | ADRENOCORTICAL.CARCINOMA |
| 2 | ATC | ANAPLASTIC.THYROID.CANCER |
| 3 | BCC | BASAL.CELL.CARCINOMA |
| 4 | BREAST.CANCER | BREAST.CANCER |
| 5 | CERVICAL.CANCER | CERVICAL.CANCER |
| 6 | COLON.CANCER | COLON.CANCER |
| 7 | EAC | ESOPHAGAL.ADENO.CARCINOMA |
| 8 | GBM | GLIOBLASTOMA.MULTIFORME |
| 9 | GIST | GASTROINTESTINAL.STROMAL.TUMOR |
| 10 | HBL | HEPATOBLASTOMA |
| 11 | HCC | HEPATOCELLULAR.CARCINOMA |
| 12 | HGBT | HIGH.GRADE.BRAIN.TUMOR |
| 13 | HL | HODGKIN.LYMPHOMA |
| 14 | LCC | NSCLC(LARGE CELL CARCINOMA) |
| 15 | LGBT | LOW.GRADE.BRAIN.TUMOR |
| 16 | MCC | MERKEL.CELL.CARCINOMA |
| 17 | MM | MULTIPLE.MYELOMA |
| 18 | NHL | NON.HODGKIN.LYMPHOMA |
| 19 | OVARIAN.CANCER | OVARIAN.CANCER |
| 20 | PANCREATIC.CANCER | PANCREATIC.CANCER |
| 21 | PNET | NEUROENDOCRINE.TUMOR |
| 22 | PPC | PERITONEAL.CANCER |
| 23 | PPGLs | PHEOCHROMOCYTOMA_PARAGANGLIOMA |
| 24 | PROSTATE.CANCER | PROSTATE.CANCER |
| 25 | RCC | RENAL.CANCER |
| 26 | RECTAL.CANCER | RECTAL.CANCER |
| 27 | SARCOMA | SARCOMA |
| 28 | SCC | NSCLC(SQUAMOUS CELL CARCINOMA) |
| 29 | SCLC | SMALL.CELL.LUNG.CANCER |
| 30 | SKIN.MELANOMA | SKIN.MELANOMA |
| 31 | STOMACH.CANCER | STOMACH.CANCER |
| 32 | UTERINE.CANCER | UTERINE.CANCER |
| 33 | UVEAL.MELANOMA | UVEAL.MELANOMA |
| 34 | WILMS.TUMOR | WILMS.TUMOR |
| 35 | cSCC | CUTANEOUS.SQUAMOUS.CELL.CARCINOMA |
| 36 | non.ATC | NON.ANAPLASTIC.THYROID.CANCER |
| 37 | non.NPC | NONNASOPHARYNGEAL.CANCER |
| 38 | ESCC | ESOPHAGAL.SQUAMOUS.CELL.CARCINOMA |
| 39 | NPC | NASOPHARYNGEAL.CANCER |
| 40 | BLC | BLADDER.CANCER |
| 41 | ADC | NSCLC(ADENOCARCINOMA) |
| 42 | BDC | BILE.DUCT.CANCER |

### Example 2. Data Preprocessing

In order to normalize the expression level of each gene in the collected data, the original data of the expression profile of all patients corresponding to each dataset produced on the same platform was normalized through methods such as SCAN, UPC ((Single-channel array normalization (SCAN) and Universal exPression Codes (UPC)), etc., and then proceeded with data cleansing such as Systematic Error, Outlier, and Missing Value.

### Example 3. Data Featurization and Model Configuration

Among 18,430 genes to be screened, genes expressed for each tumor type were primarily selected based on the tumor type of which the primary site was specified. Gene expression data attributed to the tissue was removed from the genes expressed by tumor type, and genes specifically expressed by the tumor type of which the primary site was specified were selected.

The number of genes specifically expressed by the tumor type of which the primary site is specified and the types of genes specifically expressed for each tumor type of which the primary site is specified are shown in the table below.

For the symbols of the genes listed in the table below, GEO (Gene Expression Omnibus, https://www.ncbi.nlm.nih.gov/geo/, applicable platforms: GPL570, A-AFFY-44), ArrayExpress, TCGA, ICGS, and GTEx were referenced.

TABLE 2

| | | Number of | | UNIQUE |
|---|---|---|---|---|
| Order | Cancer Type | GENES | DEG | GENE |
| 1 | ACC | 18,430 | 53 | 4 |
| 2 | ATC | 18,430 | 203 | 28 |
| 3 | BCC | 18,430 | 92 | 8 |
| 4 | BREAST.CANCER | 18,430 | 46 | 3 |
| 5 | CERVICAL.CANCER | 18,430 | 10 | 2 |
| 6 | COLON.CANCER | 18,430 | 53 | 10 |
| 7 | EAC | 18,430 | 164 | 39 |
| 8 | GBM | 18,430 | 145 | 23 |
| 9 | GIST | 18,430 | 438 | 174 |
| 10 | HBL | 18,430 | 213 | 69 |
| 11 | HCC | 18,430 | 43 | 3 |
| 12 | HGBT | 18,430 | 106 | 4 |
| 13 | HL | 18,430 | 43 | 23 |
| 14 | LCC | 18,430 | 138 | 2 |
| 15 | LGBT | 18,430 | 76 | 7 |
| 16 | MCC | 18,430 | 559 | 242 |
| 17 | MM | 18,430 | 4 | 32 |
| 18 | NHL | 18,430 | 16 | 2 |
| 19 | OVARIAN.CANCER | 18,430 | 11 | 1 |
| 20 | PANCREATIC.CANCER | 18,430 | 9 | 1 |
| 21 | PNET | 18,430 | 189 | 24 |
| 22 | PPC | 18,430 | 88 | 18 |
| 23 | PPGLs | 18,430 | 421 | 212 |
| 24 | PROSTATE.CANCER | 18,430 | 8 | 1 |
| 25 | RCC | 18,430 | 53 | 7 |
| 26 | RECTAL.CANCER | 18,430 | 140 | 44 |
| 27 | SARCOMA | 18,430 | 325 | 127 |
| 28 | SCC | 18,430 | 283 | 41 |
| 29 | SCLC | 18,430 | 319 | 44 |
| 30 | SKIN.MELANOMA | 18,430 | 108 | 25 |
| 31 | STOMACH.CANCER | 18,430 | 29 | 3 |
| 32 | UTERINE.CANCER | 18,430 | 18 | 5 |
| 33 | UVEAL.MELANOMA | 18,430 | 52 | 20 |
| 34 | WILMS.TUMOR | 18,430 | 240 | 59 |
| 35 | cSCC | 18,430 | 256 | 84 |
| 36 | non.ATC | 18,430 | 32 | 6 |
| 37 | non.NPC | 18,430 | 11 | 1 |
| 38 | ESCC | 18,430 | 13 | — |
| 39 | NPC | 18,430 | 13 | — |
| 40 | BLC | 18,430 | 8 | — |
| 41 | ADC | 18,430 | 91 | — |
| 42 | BDC | 18,430 | — | — |

DEG Selection Rule: (T-TEST < 0.001) & LOGISTIC CONCODANAT > 50 & U-TEST < 0.001 & AR > 0.3 & (−2 < LOGFOLDCHANGE < 2)

TABLE 3

| Carcinoma | Gene Names |
|---|---|
| ACC | CBLN4 |
| ACC | FMO2 |
| ACC | PTH1R |
| ACC | TH |
| ATC | ADAM12 |
| ATC | ADAMTS6 |
| ATC | ADGRE2 |
| ATC | AHNAK2 |
| ATC | ALDH1A3 |
| ATC | CCL13 |
| ATC | CLTRN |
| ATC | CRABP1 |
| ATC | CYP27C1 |
| ATC | DGKI |
| ATC | DZIP1 |
| ATC | EDN3 |
| ATC | ELOVL6 |
| ATC | GPR84 |
| ATC | HPSE |
| ATC | HRH1 |
| ATC | KCNJ13 |
| ATC | MEGF10 |
| ATC | MME |
| ATC | OTOS |
| ATC | PBX4 |
| ATC | RYR2 |
| ATC | STEAP1 |
| ATC | TBX22 |
| ATC | TCEAL2 |
| ATC | TFPI2 |
| ATC | TMEM158 |
| ATC | WSCD2 |
| BCC | ABCC12 |
| BCC | APCDD1L |
| BCC | FBN3 |
| BCC | LRP2 |
| BCC | RTN1 |
| BCC | SYNM |
| BCC | TRIM52 |
| BCC | ZNF479 |
| BREAST.CANCER | DEFB132 |
| BREAST.CANCER | SLC19A3 |
| BREAST.CANCER | UBE2T |
| CERVICAL.CANCER | GYS2 |
| CERVICAL.CANCER | SYCP2 |
| COLON.CANCER | CEL |
| COLON.CANCER | CEMIP |
| COLON.CANCER | GCG |
| COLON.CANCER | INSL5 |
| COLON.CANCER | LY6G6D |
| COLON.CANCER | S100A2 |
| COLON.CANCER | SLC30A10 |
| COLON.CANCER | TACSTD2 |
| COLON.CANCER | TCN1 |
| COLON.CANCER | UGT1A8 |
| cSCC | ACKR1 |
| cSCC | ACTA1 |
| cSCC | ACTC1 |
| cSCC | ACTG2 |
| cSCC | ADAMTS5 |
| cSCC | ADRA2A |
| cSCC | ANK2 |
| cSCC | APOBEC3A |
| cSCC | AR |
| cSCC | ARHGAP6 |
| cSCC | ARL5B |
| cSCC | ARMCX2 |
| cSCC | ATP8B4 |
| cSCC | C10orf55 |
| cSCC | CARNMT1 |
| cSCC | CCN5 |
| cSCC | CD34 |
| cSCC | CDO1 |
| cSCC | CGAS |
| cSCC | CGNL1 |
| cSCC | CHRDL1 |
| cSCC | CLEC3B |
| cSCC | CMAHP |
| cSCC | CNN1 |
| cSCC | DDIT4L |
| cSCC | DGKH |
| cSCC | EBF1 |
| cSCC | EBF2 |
| cSCC | EFHD1 |
| cSCC | EMCN |
| cSCC | EMX2 |
| cSCC | ESRRG |
| cSCC | FRZB |
| cSCC | GALNT16 |
| cSCC | GPATCH11 |
| cSCC | GPRASP1 |
| cSCC | H2AC16 |
| cSCC | H2BC13 |
| cSCC | H2BC14 |
| cSCC | H3C11 |
| cSCC | H4C5 |
| cSCC | HSD11B1 |
| cSCC | ITGB6 |
| cSCC | ITGBL1 |
| cSCC | KCNMB1 |
| cSCC | KLHL11 |
| cSCC | KNL1 |
| cSCC | LRRN4CL |
| cSCC | MACROD2 |
| cSCC | MDN1 |
| cSCC | MFAP4 |
| cSCC | MRGPRF |
| cSCC | MUC7 |
| cSCC | MYOT |
| cSCC | MYRIP |
| cSCC | OLFML1 |
| cSCC | PCSK2 |
| cSCC | PDGFD |
| cSCC | PKD2L2 |
| cSCC | PLAAT3 |
| cSCC | PLIN1 |
| cSCC | PLN |
| cSCC | PRELP |
| cSCC | PRG4 |
| cSCC | PRKAR2B |
| cSCC | RBPMS2 |
| cSCC | RECK |
| cSCC | RUNX1T1 |
| cSCC | S100A12 |
| cSCC | SH2D5 |
| cSCC | SLAIN1 |
| cSCC | SLC43A1 |
| cSCC | SLIT3 |
| cSCC | SORBS2 |
| cSCC | SPINK6 |
| cSCC | TAF13 |
| cSCC | TCEAL7 |
| cSCC | TLE2 |
| cSCC | TNIP3 |
| cSCC | VIT |
| cSCC | ZKSCAN8 |
| cSCC | ZMAT1 |
| cSCC | ZNF785 |
| cSCC | ZSCAN18 |
| EAC | ADAMTSL4 |
| EAC | ALOX12 |
| EAC | ARHGEF26 |
| EAC | BAMBI |
| EAC | BID |
| EAC | C4orf19 |
| EAC | DMBT1 |
| EAC | DNASE1L3 |
| EAC | DPT |
| EAC | DSG1 |
| EAC | EFS |
| EAC | EPB41L3 |
| EAC | FBP1 |
| EAC | FOXA3 |
| EAC | GATA6 |
| EAC | GPM6B |
| EAC | HOXB6 |
| EAC | IL1A |
| EAC | KLK12 |
| EAC | KLK13 |
| EAC | LCE3D |
| EAC | LTB4R |
| EAC | MAB21L4 |
| EAC | NECTIN3 |
| EAC | NFE2L3 |
| EAC | PAX9 |
| EAC | PRIMA1 |
| EAC | PRSS27 |
| EAC | PTPN13 |
| EAC | RBP7 |
| EAC | RORA |
| EAC | SLC16A6 |
| EAC | TIAM1 |
| EAC | TMC5 |
| EAC | TMEM40 |
| EAC | TMPRSS11B |
| EAC | VLDLR |
| EAC | ZBED2 |
| EAC | ZNF750 |
| GBM | ANXA2P2 |
| GBM | APOBEC3G |
| GBM | C11orf87 |
| GBM | CARD16 |
| GBM | CD163 |
| GBM | CD93 |
| GBM | CNGA3 |
| GBM | CRYBG1 |
| GBM | CSTA |
| GBM | DDX60L |
| GBM | LY75 |
| GBM | LY96 |
| GBM | LYZ |
| GBM | MAP3K7CL |
| GBM | MXRA5 |
| GBM | NIBAN1 |
| GBM | NNMT |
| GBM | PLP2 |
| GBM | POSTN |
| GBM | PSMB8 |
| GBM | SAMD9L |
| GBM | SERPINE1 |
| GBM | VCAM1 |
| GIST | ADCY5 |
| GIST | AKR1B10 |
| GIST | ATP10B |
| GIST | ATP4B |
| GIST | B4GALT6 |
| GIST | BBS12 |
| GIST | BHLHB9 |
| GIST | BNC2 |
| GIST | BSPRY |
| GIST | C19orf33 |
| GIST | C1QTNF2 |
| GIST | C1orf216 |
| GIST | C6orf58 |
| GIST | CAND2 |
| GIST | CARF |
| GIST | CBLIF |
| GIST | CDH1 |
| GIST | CHIA |
| GIST | CLCA1 |
| GIST | CLMN |
| GIST | CPA2 |
| GIST | CSPG4 |
| GIST | CSRNP3 |
| GIST | CXADR |
| GIST | CYP2C9 |
| GIST | CYP2S1 |
| GIST | CYS1 |
| GIST | DCAF12L2 |
| GIST | DIRAS3 |
| GIST | DSC2 |
| GIST | EID3 |
| GIST | ELF3 |
| GIST | EPB41L4B |
| GIST | ERBB3 |
| GIST | ESRP1 |
| GIST | ESRP2 |
| GIST | F2RL1 |
| GIST | F2RL2 |
| GIST | FA2H |
| GIST | FAM110B |
| GIST | FAM229B |
| GIST | FAM3D |
| GIST | FBXL2 |
| GIST | FGF2 |
| GIST | FUT2 |
| GIST | FUT3 |
| GIST | FXYD3 |
| GIST | GABRA2 |
| GIST | GALE |
| GIST | GCNT3 |
| GIST | GKN1 |
| GIST | GPA33 |
| GIST | GPR37 |
| GIST | GPRC5A |
| GIST | GPX2 |
| GIST | GREM2 |
| GIST | GSDMB |
| GIST | GSDME |
| GIST | GUCY2C |
| GIST | HECW2 |
| GIST | HOXA2 |
| GIST | HSD11B2 |
| GIST | IMPA2 |
| GIST | INTU |
| GIST | IRF6 |
| GIST | ISL2 |
| GIST | ISLR |
| GIST | KCNE4 |
| GIST | KCNJ8 |
| GIST | KCNK3 |
| GIST | KLK11 |
| GIST | LCA5 |
| GIST | LCN2 |
| GIST | LGALS4 |
| GIST | LIPH |
| GIST | LPAR4 |
| GIST | LRCH2 |
| GIST | LRRC3B |
| GIST | LRRC66 |
| GIST | LSAMP |
| GIST | LY6H |
| GIST | MAGEL2 |
| GIST | MAGI2 |
| GIST | MAL2 |
| GIST | MAP3K21 |
| GIST | MAPK10 |
| GIST | MAPK13 |
| GIST | MGST1 |
| GIST | MPP6 |
| GIST | MRAP2 |
| GIST | MT1M |
| GIST | MUC1 |
| GIST | MUC4 |
| GIST | MUC6 |
| GIST | MYO1A |
| GIST | MYO5B |
| GIST | N6AMT1 |
| GIST | NAV3 |
| GIST | NKX3-2 |
| GIST | NLGN4Y |
| GIST | NPFFR2 |
| GIST | NRIP3 |
| GIST | NRK |
| GIST | OBSCN |
| GIST | OLFM4 |
| GIST | OSGIN2 |
| GIST | OVOL2 |
| GIST | PALD1 |
| GIST | PCDHB15 |
| GIST | PCDHB3 |
| GIST | PCDHB5 |
| GIST | PDE10A |
| GIST | PDE4C |
| GIST | PI3 |
| GIST | PIGR |
| GIST | PIK3CG |
| GIST | PKP2 |
| GIST | PLA2G4C |
| GIST | PLEKHA7 |
| GIST | PLEKHH1 |
| GIST | PLPP2 |
| GIST | PLS1 |
| GIST | PLXDC1 |
| GIST | PLXDC2 |
| GIST | POU2AF1 |
| GIST | PPL |
| GIST | PRICKLE1 |
| GIST | PRSS16 |
| GIST | PTPRR |
| GIST | RAB25 |
| GIST | REG1A |
| GIST | REG4 |
| GIST | RNF128 |
| GIST | RNF24 |
| GIST | SAMD13 |
| GIST | SCARA3 |
| GIST | SCIN |
| GIST | SEMA3A |
| GIST | SERINC2 |
| GIST | SERPINB5 |
| GIST | SGCD |
| GIST | SLC26A3 |
| GIST | SLC28A2 |
| GIST | SLC44A3 |
| GIST | SLC51B |
| GIST | SMCO3 |
| GIST | SOX9 |
| GIST | SPINK5 |
| GIST | SPINT1 |
| GIST | SPTSSB |
| GIST | STYK1 |
| GIST | SULT1B1 |
| GIST | TAFA4 |
| GIST | TC2N |
| GIST | TFF3 |
| GIST | TMEM125 |
| GIST | TMEM171 |
| GIST | TMEM231 |
| GIST | TMPRSS2 |
| GIST | TNFRSF11A |
| GIST | TNFRSF17 |
| GIST | TRIM23 |
| GIST | TRPC1 |
| GIST | TRPC3 |
| GIST | TTC39A |
| GIST | UGT2B15 |
| GIST | VNN1 |
| GIST | VSIG1 |
| GIST | WDFY3-AS2 |
| GIST | ZC3H12D |
| GIST | ZNF135 |
| GIST | ZNF415 |
| GIST | ZNF542P |
| GIST | ZNF569 |
| HBL | ABCB11 |
| HBL | ARID3A |
| HBL | ASPSCR1 |
| HBL | BCL11A |
| HBL | BEND5 |
| HBL | C9 |
| HBL | CGREF1 |
| HBL | CLEC1B |
| HBL | COLEC12 |
| HBL | CRP |
| HBL | CYP26A1 |
| HBL | CYP2B6 |
| HBL | DEFA5 |
| HBL | DUSP9 |
| HBL | EDDM3A |
| HBL | ERVMER34-1 |
| HBL | FAM217B |
| HBL | FCN2 |
| HBL | FETUB |
| HBL | FGF20 |
| HBL | GABRB1 |
| HBL | GNAL |
| HBL | GPLD1 |
| HBL | GXYLT2 |
| HBL | HMGA2 |
| HBL | HPGD |
| HBL | HSDL1 |
| HBL | IDO2 |
| HBL | IGDCC3 |
| HBL | IGF2BP1 |
| HBL | IGF2BP2 |
| HBL | ITGA2 |
| HBL | LIN28B |
| HBL | LINC01549 |
| HBL | MAP7D2 |
| HBL | MUCL1 |
| HBL | NAALAD2 |
| HBL | NAT2 |
| HBL | NKD1 |
| HBL | OLR1 |
| HBL | OXCT1 |
| HBL | PGAP1 |
| HBL | PGC |
| HBL | PPP1R9A |
| HBL | PRTG |
| HBL | QPCT |
| HBL | REG3A |
| HBL | RFX6 |
| HBL | SACS |
| HBL | SDS |
| HBL | SEC14L4 |
| HBL | SELE |
| HBL | SHISA6 |
| HBL | SLC17A4 |
| HBL | SLC7A11 |
| HBL | SPDL1 |
| HBL | SRD5A2 |
| HBL | SSUH2 |
| HBL | ST18 |
| HBL | TAF1L |
| HBL | TBX15 |
| HBL | TRH |
| HBL | TRPM8 |
| HBL | TSPAN5 |
| HBL | USP27X |
| HBL | ZG16 |
| HBL | ZNF594 |
| HBL | ZRANB3 |
| HBL | ZSWIM5 |
| HCC | ADGRG7 |
| HCC | CXCL14 |
| HCC | OIT3 |
| HGBT | AFDN-DT |
| HGBT | CREB3L4 |
| HGBT | HFM1 |
| HGBT | OTX2 |
| HL | ANKDD1A |
| HL | C1orf115 |
| HL | DSP |
| HL | EPHA2 |
| HL | FHDC1 |
| HL | GABBR1 |
| HL | GPR182 |
| HL | GZMH |
| HL | HOXA5 |
| HL | L3MBTL3 |
| HL | LIMCH1 |
| HL | LOC654780 |
| HL | NINL |
| HL | PCDH9 |
| HL | PDE2A |
| HL | PLCXD3 |
| HL | PRKY |
| HL | PTGR1 |
| HL | SH3BGRL2 |
| HL | STAB2 |
| HL | TAGLN3 |
| HL | TIE1 |
| HL | WHRN |
| LCC | CFAP53 |
| LCC | SLC6A4 |
| LGBT | CALCRL |
| LGBT | MAP3K8 |
| LGBT | MORC4 |
| LGBT | PTGR2 |
| LGBT | TNFAIP8 |
| LGBT | TNFRSF11B |
| LGBT | TTC30B |
| MCC | AADACL2 |
| MCC | ABCA12 |
| MCC | ABCA6 |
| MCC | ABLIM3 |
| MCC | ACP3 |
| MCC | ACSM3 |
| MCC | ACSS2 |
| MCC | ADGRG6 |
| MCC | AHCYL2 |
| MCC | AKNAD1 |
| MCC | AKR1C3 |
| MCC | ALDH3A1 |
| MCC | ALDH3B2 |
| MCC | ALOX12B |
| MCC | ALOXE3 |
| MCC | AMER1 |
| MCC | AMER2 |
| MCC | ANKRD29 |
| MCC | ANO5 |
| MCC | ANXA3 |
| MCC | ANXA9 |
| MCC | APLF |
| MCC | AQP9 |
| MCC | ARG1 |
| MCC | ARHGAP42 |
| MCC | ARHGEF37 |
| MCC | ATP10A |
| MCC | ATP6V1C2 |
| MCC | AVPI1 |
| MCC | AWAT1 |
| MCC | BEAN1 |
| MCC | BEST3 |
| MCC | BPIFC |
| MCC | BRAF |
| MCC | BTBD16 |
| MCC | BTD |
| MCC | C11orf45 |
| MCC | C3orf52 |
| MCC | C5orf46 |
| MCC | CA6 |
| MCC | CAPN3 |
| MCC | CARD18 |
| MCC | CCDC9B |
| MCC | CCL27 |
| MCC | CD1E |
| MCC | CDH19 |
| MCC | CDHR1 |
| MCC | CDR1 |
| MCC | CDSN |
| MCC | CHI3L2 |
| MCC | CNGA1 |
| MCC | CNTN2 |
| MCC | COL17A1 |
| MCC | CTSG |
| MCC | CXCR2 |
| MCC | CYP2E1 |
| MCC | CYP4F22 |
| MCC | CYP4F8 |
| MCC | CYSRT1 |
| MCC | DCT |
| MCC | DCUN1D1 |
| MCC | DEGS2 |
| MCC | DGKA |
| MCC | DIAPH2 |
| MCC | DSC1 |
| MCC | DUSP26 |
| MCC | EGLN3 |
| MCC | ELF5 |
| MCC | ENTPD3 |
| MCC | EPN3 |
| MCC | EPS8L1 |
| MCC | ERC2 |
| MCC | ESYT3 |
| MCC | ETFBKMT |
| MCC | EVPL |
| MCC | EXPH5 |
| MCC | FAH |
| MCC | FEM1B |
| MCC | FMO4 |
| MCC | GABRE |
| MCC | GAN |
| MCC | GFI1 |
| MCC | GFPT2 |
| MCC | GJB3 |
| MCC | GPR34 |
| MCC | GPRIN2 |
| MCC | GRAMD1C |
| MCC | GRHL1 |
| MCC | GULP1 |
| MCC | HAL |
| MCC | HDC |
| MCC | HS3ST6 |
| MCC | IGSF10 |
| MCC | IL17RD |
| MCC | IL22RA1 |
| MCC | IL33 |
| MCC | ISM1 |
| MCC | ITPR2 |
| MCC | KCNH6 |
| MCC | KCNK5 |
| MCC | KCNK7 |
| MCC | KCTD11 |
| MCC | KCTD21 |
| MCC | KLF8 |
| MCC | KLK1 |
| MCC | KLK10 |
| MCC | KLK8 |
| MCC | KRT2 |
| MCC | KRT27 |
| MCC | KRT31 |
| MCC | KRT73 |
| MCC | KRT74 |
| MCC | KRT77 |
| MCC | KRTAP11-1 |
| MCC | KRTAP2-1 |
| MCC | KRTAP3-1 |
| MCC | KRTAP4-7 |
| MCC | LAMB4 |
| MCC | LCE2B |
| MCC | LEPR |
| MCC | LHX3 |
| MCC | LIFR |
| MCC | LPAR5 |
| MCC | LY6G6C |
| MCC | LYNX1 |
| MCC | LYPD6B |
| MCC | MAB21L3 |
| MCC | MAN1A2 |
| MCC | MATN2 |
| MCC | MFAP3L |
| MCC | MICA |
| MCC | MID2 |
| MCC | MIR99AHG |
| MCC | MLANA |
| MCC | MMP28 |
| MCC | MPP7 |
| MCC | MPZ |
| MCC | MS4A2 |
| MCC | MST1R |
| MCC | MTMR11 |
| MCC | MYEOV |
| MCC | NAA40 |
| MCC | NDNF |
| MCC | NECTIN4 |
| MCC | NEUROD2 |
| MCC | NEXN |
| MCC | NIM1K |
| MCC | NIPAL2 |
| MCC | NIPAL4 |
| MCC | NLRP1 |
| MCC | NPAS2 |
| MCC | NPTXR |
| MCC | NTN4 |
| MCC | NTRK2 |
| MCC | OBP2B |
| MCC | PCDH7 |
| MCC | PEX11A |
| MCC | PHYHIP |
| MCC | PITPNM3 |
| MCC | PLA2G3 |
| MCC | PLA2G4F |
| MCC | PLD1 |
| MCC | PLEKHG1 |
| MCC | PMEL |
| MCC | PNLIPRP3 |
| MCC | POU2F3 |
| MCC | POU3F2 |
| MCC | PPFIBP1 |
| MCC | PPP1R13L |
| MCC | PPP1R3B |
| MCC | PRSS12 |
| MCC | PSAPL1 |
| MCC | PSORS1C2 |
| MCC | PTGES |
| MCC | PTK6 |
| MCC | PTPN21 |
| MCC | PXK |
| MCC | RFTN2 |
| MCC | RGN |
| MCC | RHOJ |
| MCC | RHOV |
| MCC | RIMS2 |
| MCC | RNASE4 |
| MCC | RNF39 |
| MCC | RPTN |
| MCC | RSPO1 |
| MCC | RUNDC3B |
| MCC | SBSPON |
| MCC | SCGN |
| MCC | SCUBE2 |
| MCC | SELP |
| MCC | SEMA3G |
| MCC | SEMA4G |
| MCC | SERHL2 |
| MCC | SERPINA12 |
| MCC | SERPINA3 |
| MCC | SERPINA5 |
| MCC | SERPINB7 |
| MCC | SERPINB8 |
| MCC | SGPP2 |
| MCC | SH3RF2 |
| MCC | SLC20A2 |
| MCC | SLC25A18 |
| MCC | SLC28A3 |
| MCC | SLC2A12 |
| MCC | SLC39A2 |
| MCC | SLC5A1 |
| MCC | SLC9A9 |
| MCC | SMAD5-AS1 |
| MCC | SNCA |
| MCC | SNTB1 |
| MCC | SNX21 |
| MCC | SOSTDC1 |
| MCC | SPTLC3 |
| MCC | STARD5 |
| MCC | STK32B |
| MCC | TAFA2 |
| MCC | TG |
| MCC | THSD7B |
| MCC | TLR3 |
| MCC | TLR5 |
| MCC | TMEM108 |
| MCC | TMEM144 |
| MCC | TMEM74 |
| MCC | TMEM79 |
| MCC | TP53AIP1 |
| MCC | TRIM7 |
| MCC | TRPM1 |
| MCC | TYR |
| MCC | UEVLD |
| MCC | VIPR1 |
| MCC | VSNL1 |
| MCC | WFDC12 |
| MCC | WFDC3 |
| MCC | WFDC5 |
| MCC | WLS |
| MCC | ZNF204P |
| MCC | ZNF224 |
| MCC | ZNF563 |
| MCC | ZNF600 |
| MCC | ZNF677 |
| MCC | ZNF846 |
| MM | MOSPD2 |
| MM | RNASEL |
| MM | ZNF486 |
| NHL | GINS3 |
| NHL | NEK2 |
| non.ATC | ARHGAP36 |
| non.ATC | DCSTAMP |
| non.ATC | FAM20A |
| non.ATC | GABRB2 |
| non.ATC | RXRG |
| non.ATC | RYR1 |
| non.NPC | IL24 |
| OVARIAN.CANCER | CTCFL |
| PANCREATIC.CANCER | LEMD1 |
| PNET | ARPP21 |
| PNET | CACNG3 |
| PNET | CCDC15 |
| PNET | CHAC2 |
| PNET | ERMN |
| PNET | GABRG1 |
| PNET | GTSE1 |
| PNET | IPCEF1 |
| PNET | MASTL |
| PNET | MCM3AP-AS1 |
| PNET | MFAP2 |
| PNET | MOBP |
| PNET | MOG |
| PNET | RFC5 |
| PNET | SAAL1 |
| PNET | SEC14L5 |
| PNET | SLC39A12 |
| PNET | SOWAHC |
| PNET | TMEM155 |
| PNET | TTF2 |
| PNET | UNC13C |
| PNET | WDR76 |
| PNET | ZNF764 |
| PNET | ZNF814 |
| PPC | ACVR1C |
| PPC | ADGRL3 |
| PPC | CCDC178 |
| PPC | CHST7 |
| PPC | CIDEA |
| PPC | COL6A6 |
| PPC | COLGALT2 |
| PPC | FBLN7 |
| PPC | GPC3 |
| PPC | KCNN3 |
| PPC | LDB3 |
| PPC | MIR1-1HG-AS1 |
| PPC | P2RY14 |
| PPC | PAGE4 |
| PPC | PNOC |
| PPC | PPP1R1A |
| PPC | SOX7 |
| PPC | WFDC1 |
| PPGLs | ADAMTS19 |
| PPGLs | ADCYAP1R1 |
| PPGLs | ADGRA1 |
| PPGLs | ADGRB2 |
| PPGLs | ADORA3 |
| PPGLs | AK4 |
| PPGLs | AP3B2 |
| PPGLs | ARAP2 |
| PPGLs | ARC |
| PPGLs | ASB4 |
| PPGLs | ASPHD2 |
| PPGLs | ASTN2 |
| PPGLs | ATP1A3 |
| PPGLs | ATP4A |
| PPGLs | ATP6V1G2 |
| PPGLs | B3GAT1 |
| PPGLs | BEGAIN |
| PPGLs | BICD1 |
| PPGLs | BMP7 |
| PPGLs | BRINP1 |
| PPGLs | C14orf39 |
| PPGLs | C1QL1 |
| PPGLs | CA10 |
| PPGLs | CACNA1B |
| PPGLs | CACNA2D3 |
| PPGLs | CADM2 |
| PPGLs | CALN1 |
| PPGLs | CALY |
| PPGLs | CAMK2B |
| PPGLs | CAMK4 |
| PPGLs | CBLN3 |
| PPGLs | CCNA1 |
| PPGLs | CCR10 |
| PPGLs | CCSER1 |
| PPGLs | CD200 |
| PPGLs | CDH18 |
| PPGLs | CDK5R2 |
| PPGLs | CELF6 |
| PPGLs | CELSR3 |
| PPGLs | CHRNB4 |
| PPGLs | CKMT2 |
| PPGLs | CLCN4 |
| PPGLs | CNKSR2 |
| PPGLs | CNNM1 |
| PPGLs | CPLX2 |
| PPGLs | CREB5 |
| PPGLs | CTNNA2 |
| PPGLs | CYP11B2 |
| PPGLs | DDC |
| PPGLs | DDX25 |
| PPGLs | DGKB |
| PPGLs | DHRS2 |
| PPGLs | DISP2 |
| PPGLs | DLX1 |
| PPGLs | DOK5 |
| PPGLs | DRD2 |
| PPGLs | EGR4 |
| PPGLs | FAM133A |
| PPGLs | FAM174B |
| PPGLs | FBXO16 |
| PPGLs | FEV |
| PPGLs | FLVCR2 |
| PPGLs | FMN2 |
| PPGLs | FMO1 |
| PPGLs | GABRG2 |
| PPGLs | GALNT14 |
| PPGLs | GALNT18 |
| PPGLs | GALR1 |
| PPGLs | GAP43 |
| PPGLs | GATA3 |
| PPGLs | GCNA |
| PPGLs | GDAP1 |
| PPGLs | GFRA3 |
| PPGLs | GLRB |
| PPGLs | GNG3 |
| PPGLs | GPR176 |
| PPGLs | GPR22 |
| PPGLs | GRIA4 |
| PPGLs | GRIP1 |
| PPGLs | HAND1 |
| PPGLs | HCN1 |
| PPGLs | HMGCLL1 |
| PPGLs | HOXC10 |
| PPGLs | HOXC9 |
| PPGLs | HPCAL4 |
| PPGLs | HS3ST2 |
| PPGLs | IL1RL1 |
| PPGLs | INS |
| PPGLs | INSM2 |
| PPGLs | ISL1 |
| PPGLs | JAKMIP1 |
| PPGLs | JPH4 |
| PPGLs | KCNB1 |
| PPGLs | KCNH2 |
| PPGLs | KCNJ6 |
| PPGLs | KCNK12 |
| PPGLs | KCNK2 |
| PPGLs | KCNQ5 |
| PPGLs | KCTD16 |
| PPGLs | KIAA1841 |
| PPGLs | KIF1A |
| PPGLs | KLHL4 |
| PPGLs | L1CAM |
| PPGLs | LAMA2 |
| PPGLs | LAYN |
| PPGLs | LINGO2 |
| PPGLs | LMO1 |
| PPGLs | LRRC39 |
| PPGLs | MAB21L2 |
| PPGLs | MAMSTR |
| PPGLs | MAPT |
| PPGLs | MARCHF11 |
| PPGLs | MARCHF4 |
| PPGLs | MARK1 |
| PPGLs | MBOAT2 |
| PPGLs | MC2R |
| PPGLs | MCF2 |
| PPGLs | MCOLN2 |
| PPGLs | MELTF |
| PPGLs | MINAR1 |
| PPGLs | MIR7-3HG |
| PPGLs | MRAP |
| PPGLs | MYT1 |
| PPGLs | MYT1L |
| PPGLs | NDUFA4L2 |
| PPGLs | NLGN4X |
| PPGLs | NMNAT2 |
| PPGLs | NROB1 |
| PPGLs | NRXN1 |
| PPGLs | NTRK1 |
| PPGLs | OPRK1 |
| PPGLs | OSBPL3 |
| PPGLs | OSR2 |
| PPGLs | PCBP3 |
| PPGLs | PCLO |
| PPGLs | PDE3A |
| PPGLs | PDLIM4 |
| PPGLs | PHOSPHO2 |
| PPGLs | PHOX2A |
| PPGLs | PHOX2B |
| PPGLs | PKIA |
| PPGLs | PLXNA2 |
| PPGLs | PPP2R2C |
| PPGLs | PRKCD |
| PPGLs | PRLHR |
| PPGLs | PRPH |
| PPGLs | PTGER2 |
| PPGLs | PTGS1 |
| PPGLs | PTPRN |
| PPGLs | PTPRO |
| PPGLs | RAB15 |
| PPGLs | RAB27B |
| PPGLs | RAB33A |
| PPGLs | RAB38 |
| PPGLs | RAB6B |
| PPGLs | RASD2 |
| PPGLs | RASEF |
| PPGLs | RBM47 |
| PPGLs | RD3 |
| PPGLs | REEP2 |
| PPGLs | RET |
| PPGLs | RIIAD1 |
| PPGLs | RIMS3 |
| PPGLs | RPH3A |
| PPGLs | RUNDC3A |
| PPGLs | SCN3B |
| PPGLs | SCN9A |
| PPGLs | SEPTIN3 |
| PPGLs | SEZ6L |
| PPGLs | SGIP1 |
| PPGLs | SHOC1 |
| PPGLs | SIDT1 |
| PPGLs | SIGLEC11 |
| PPGLs | SLC12A5 |
| PPGLs | SLC18A1 |
| PPGLs | SLC24A2 |
| PPGLs | SLC35F3 |
| PPGLs | SLC38A11 |
| PPGLs | SLC51A |
| PPGLs | SLC6A2 |
| PPGLs | SLC6A9 |
| PPGLs | SLC8A2 |
| PPGLs | SOGA1 |
| PPGLs | SPAG1 |
| PPGLs | SPDYE1 |
| PPGLs | SRD5A1 |
| PPGLs | SSX2IP |
| PPGLs | ST8SIA3 |
| PPGLs | ST8SIA5 |
| PPGLs | STMN4 |
| PPGLs | SULT2A1 |
| PPGLs | SVOP |
| PPGLs | SYN1 |
| PPGLs | SYNGR3 |
| PPGLs | SYNPR |
| PPGLs | SYT14 |
| PPGLs | TCP11L2 |
| PPGLs | TDRKH |
| PPGLs | TMEM130 |
| PPGLs | TMEM145 |
| PPGLs | TMIE |
| PPGLs | TPD52 |
| PPGLs | TPPP |
| PPGLs | TTLL7 |
| PPGLs | TUBB4A |
| PPGLs | UNC5A |
| PPGLs | UNC79 |
| PPGLs | VEPH1 |
| PPGLs | WDR17 |
| PPGLs | YPEL4 |
| PPGLs | ZBTB6 |
| PPGLs | ZFR2 |
| PROSTATE.CANCER | TDRD1 |
| RCC | CRYAA |
| RCC | GPC5 |
| RCC | IDO1 |
| RCC | MTTP |
| RCC | NPHS2 |
| RCC | SFRP1 |
| RCC | SPAG4 |
| RECTAL.CANCER | ADGRF5 |
| RECTAL.CANCER | AGT |
| RECTAL.CANCER | BRCA2 |
| RECTAL.CANCER | C4BPA |
| RECTAL.CANCER | CCDC113 |
| RECTAL.CANCER | CENPN |
| RECTAL.CANCER | CEP72 |
| RECTAL.CANCER | CEP83 |
| RECTAL.CANCER | COL12A1 |
| RECTAL.CANCER | DDX55 |
| RECTAL.CANCER | DNMT3B |
| RECTAL.CANCER | ERCC6L |
| RECTAL.CANCER | ETV4 |
| RECTAL.CANCER | FCGR3B |
| RECTAL.CANCER | FIGNL1 |
| RECTAL.CANCER | FPR1 |
| RECTAL.CANCER | GAS2 |
| RECTAL.CANCER | GPT2 |
| RECTAL.CANCER | GZMB |
| RECTAL.CANCER | HAUS6 |
| RECTAL.CANCER | IFI44L |
| RECTAL.CANCER | JADE3 |
| RECTAL.CANCER | KIAA0895 |
| RECTAL.CANCER | MACC1 |
| RECTAL.CANCER | MARS2 |
| RECTAL.CANCER | NAA25 |
| RECTAL.CANCER | NANP |
| RECTAL.CANCER | NUP155 |
| RECTAL.CANCER | NUP62CL |
| RECTAL.CANCER | PDCD2L |
| RECTAL.CANCER | PIR |
| RECTAL.CANCER | PLAU |
| RECTAL.CANCER | RFWD3 |
| RECTAL.CANCER | SKA3 |
| RECTAL.CANCER | SLC35E4 |
| RECTAL.CANCER | SLC38A5 |
| RECTAL.CANCER | SLC6A20 |
| RECTAL.CANCER | SLC7A5 |
| RECTAL.CANCER | TBC1D31 |
| RECTAL.CANCER | TNFSF15 |
| RECTAL.CANCER | UBE3D |
| RECTAL.CANCER | UTP15 |
| RECTAL.CANCER | WNT2 |
| RECTAL.CANCER | ZNF280C |
| SARCOMA | ABRA |
| SARCOMA | ACOT7 |
| SARCOMA | ACTN3 |
| SARCOMA | ADAM10 |
| SARCOMA | ANKRD2 |
| SARCOMA | ANKRD23 |
| SARCOMA | AQP4 |
| SARCOMA | ARL4C |
| SARCOMA | ATP1B4 |
| SARCOMA | BCL11B |
| SARCOMA | BMP2K |
| SARCOMA | C10orf71 |
| SARCOMA | C18orf54 |
| SARCOMA | C3orf14 |
| SARCOMA | CACNA1S |
| SARCOMA | CCDC137 |
| SARCOMA | CCL4 |
| SARCOMA | CCNB2 |
| SARCOMA | CDNF |
| SARCOMA | CEP152 |
| SARCOMA | CLIC5 |
| SARCOMA | CLIP2 |
| SARCOMA | CXCR4 |
| SARCOMA | DHRS7C |
| SARCOMA | DUSP13 |
| SARCOMA | ECT2 |
| SARCOMA | EGR2 |
| SARCOMA | EMILIN1 |
| SARCOMA | FANCG |
| SARCOMA | FBXO40 |
| SARCOMA | FPR3 |
| SARCOMA | GAS2L3 |
| SARCOMA | GLMP |
| SARCOMA | GPR183 |
| SARCOMA | HJV |
| SARCOMA | IDI2 |
| SARCOMA | ITGA4 |
| SARCOMA | KBTBD12 |
| SARCOMA | KCNA7 |
| SARCOMA | KIF20B |
| SARCOMA | KIF2A |
| SARCOMA | KLHL40 |
| SARCOMA | LINC00310 |
| SARCOMA | LIPI |
| SARCOMA | LMNB2 |
| SARCOMA | LMOD3 |
| SARCOMA | LRRC37A3 |
| SARCOMA | LSMEM1 |
| SARCOMA | MERTK |
| SARCOMA | MFHAS1 |
| SARCOMA | MICB |
| SARCOMA | MYF6 |
| SARCOMA | MYH1 |
| SARCOMA | MYH4 |
| SARCOMA | MYH6 |
| SARCOMA | MYLK3 |
| SARCOMA | NAT1 |
| SARCOMA | NKX2-2 |
| SARCOMA | NRAP |
| SARCOMA | NUDT11 |
| SARCOMA | ORC6 |
| SARCOMA | P2RY2 |
| SARCOMA | P3H1 |
| SARCOMA | PABPC1L |
| SARCOMA | PAPPA |
| SARCOMA | PARPBP |
| SARCOMA | PCDH17 |
| SARCOMA | PFKFB1 |
| SARCOMA | PHETA2 |
| SARCOMA | PIEZO2 |
| SARCOMA | PLAUR |
| SARCOMA | PLPP5 |
| SARCOMA | PNMA2 |
| SARCOMA | PPDPFL |
| SARCOMA | PPP1R3A |
| SARCOMA | PRKAG3 |
| SARCOMA | PRKCQ |
| SARCOMA | PRMT6 |
| SARCOMA | PRR5L |
| SARCOMA | PRSS35 |
| SARCOMA | PSD3 |
| SARCOMA | PTPN22 |
| SARCOMA | PTTG1 |
| SARCOMA | PYGM |
| SARCOMA | RAI14 |
| SARCOMA | RBBP8 |
| SARCOMA | RBM11 |
| SARCOMA | RGS1 |
| SARCOMA | RNF182 |
| SARCOMA | ROR1 |
| SARCOMA | RPL3L |
| SARCOMA | RUBCNL |
| SARCOMA | RUNX3 |
| SARCOMA | SAMSN1 |
| SARCOMA | SCG2 |
| SARCOMA | SCLT1 |
| SARCOMA | SDC1 |
| SARCOMA | SMC2 |
| SARCOMA | SMCO1 |
| SARCOMA | SPAG5 |
| SARCOMA | SPIN4 |
| SARCOMA | SQLE |
| SARCOMA | SYNPO2L |
| SARCOMA | SYPL2 |
| SARCOMA | TACC3 |
| SARCOMA | TBC1D8B |
| SARCOMA | TECRL |
| SARCOMA | TK1 |
| SARCOMA | TLCD3A |
| SARCOMA | TLR1 |
| SARCOMA | TMED3 |
| SARCOMA | TMEM182 |
| SARCOMA | TMEM200A |
| SARCOMA | TMOD4 |
| SARCOMA | TOX2 |
| SARCOMA | TRDN |
| SARCOMA | TRIM63 |
| SARCOMA | TSHZ3 |
| SARCOMA | TYMS |
| SARCOMA | UBE2C |
| SARCOMA | UCP3 |
| SARCOMA | UNC45B |
| SARCOMA | ZNF136 |
| SARCOMA | ZNF430 |
| SARCOMA | ZNF667 |
| SARCOMA | ZWILCH |
| SARCOMA | ZWINT |
| SCC | ADAM23 |
| SCC | AK7 |
| SCC | AK9 |
| SCC | C12orf56 |
| SCC | C2orf73 |
| SCC | CALML3 |
| SCC | CCDC148 |
| SCC | CCDC151 |
| SCC | CCDC30 |
| SCC | CFAP206 |
| SCC | CNTD1 |
| SCC | DCDC2 |
| SCC | DNAH7 |
| SCC | DRC1 |
| SCC | DSG3 |
| SCC | EFHC2 |
| SCC | ERBB4 |
| SCC | FAM149A |
| SCC | FAM184A |
| SCC | FBXO15 |
| SCC | FYB2 |
| SCC | IL36G |
| SCC | KRT13 |
| SCC | KRT14 |
| SCC | KRT16 |
| SCC | KRT6A |
| SCC | KRT6B |
| SCC | MAATS1 |
| SCC | MAGEA11 |
| SCC | MAGEA4 |
| SCC | NSUN7 |
| SCC | PCDH19 |
| SCC | RP1 |
| SCC | SLC22A16 |
| SCC | SPATA17 |
| SCC | SPATA4 |
| SCC | SPATA6 |
| SCC | SPRR1A |
| SCC | SPRR2A |
| SCC | STK33 |
| SCC | UBXN10 |
| SCLC | ABCA13 |
| SCLC | ADGB |
| SCLC | ADRB1 |
| SCLC | ALDH3B1 |
| SCLC | ANG |
| SCLC | ASCL1 |
| SCLC | BPIFB1 |
| SCLC | CCDC170 |
| SCLC | CCDC186 |
| SCLC | CCDC68 |
| SCLC | CCNE1 |
| SCLC | CDH26 |
| SCLC | CNTNAP2 |
| SCLC | CX3CR1 |
| SCLC | DLX5 |
| SCLC | DNAH12 |
| SCLC | ELOVL2 |
| SCLC | ESPL1 |
| SCLC | FCN1 |
| SCLC | FILIP1 |
| SCLC | FLACC1 |
| SCLC | FOSB |
| SCLC | GNA14 |
| SCLC | GPIHBP1 |
| SCLC | HHLA2 |
| SCLC | KCNH8 |
| SCLC | LHX2 |
| SCLC | MANEAL |
| SCLC | MCEMP1 |
| SCLC | MUC5B |
| SCLC | MYCT1 |
| SCLC | ODF3B |
| SCLC | PRDM13 |
| SCLC | PRICKLE2 |
| SCLC | PROX1 |
| SCLC | RBM43 |
| SCLC | RRAD |
| SCLC | RSPO2 |
| SCLC | SERPINB3 |
| SCLC | SLC16A5 |
| SCLC | TCF21 |
| SCLC | TMEM71 |
| SCLC | TRPC6 |
| SCLC | VMO1 |
| SKIN.MELANOMA | CPN1 |
| SKIN.MELANOMA | ENTHD1 |
| SKIN.MELANOMA | FCRLA |
| SKIN.MELANOMA | FSTL5 |
| SKIN.MELANOMA | GDF15 |
| SKIN.MELANOMA | KRT79 |
| SKIN.MELANOMA | KRTAP1-1 |
| SKIN.MELANOMA | KRTAP1-3 |
| SKIN.MELANOMA | KRTAP2-4 |
| SKIN.MELANOMA | KRTAP3-3 |
| SKIN.MELANOMA | KRTAP4-4 |
| SKIN.MELANOMA | KRTAP9-3 |
| SKIN.MELANOMA | KRTAP9-4 |
| SKIN.MELANOMA | LINC00518 |
| SKIN.MELANOMA | MAGEC1 |
| SKIN.MELANOMA | MAGEC2 |
| SKIN.MELANOMA | PLA1A |
| SKIN.MELANOMA | RASSF10 |
| SKIN.MELANOMA | RNASE7 |
| SKIN.MELANOMA | SHANK2 |
| SKIN.MELANOMA | SLC45A2 |
| SKIN.MELANOMA | SLC6A15 |
| SKIN.MELANOMA | TPTE |
| SKIN.MELANOMA | TRIM51 |
| SKIN.MELANOMA | ZNF280B |
| STOMACH.CANCER | FNDC1 |
| STOMACH.CANCER | MS4A12 |
| STOMACH.CANCER | SPP1 |
| UTERINE.CANCER | JCHAIN |
| UTERINE.CANCER | KANK4 |
| UTERINE.CANCER | MMP26 |
| UTERINE.CANCER | PAEP |
| UTERINE.CANCER | RAMP2 |
| UVEAL.MELANOMA | ANKRD34A |
| UVEAL.MELANOMA | BAG2 |
| UVEAL.MELANOMA | CCDC177 |
| UVEAL.MELANOMA | CPNE6 |
| UVEAL.MELANOMA | DEFB119 |
| UVEAL.MELANOMA | FEZF2 |
| UVEAL.MELANOMA | GRIA3 |
| UVEAL.MELANOMA | IQCG |
| UVEAL.MELANOMA | LNX1 |
| UVEAL.MELANOMA | MDGA2 |
| UVEAL.MELANOMA | METTL1 |
| UVEAL.MELANOMA | PAK5 |
| UVEAL.MELANOMA | PCAT4 |
| UVEAL.MELANOMA | REPS2 |
| UVEAL.MELANOMA | RLN2 |
| UVEAL.MELANOMA | SCN1A |
| UVEAL.MELANOMA | SLC24A4 |
| UVEAL.MELANOMA | SLC35F4 |
| UVEAL.MELANOMA | SLITRK6 |
| UVEAL.MELANOMA | ZNF804A |
| WILMS.TUMOR | ACMSD |
| WILMS.TUMOR | ADH6 |
| WILMS.TUMOR | AGXT2 |
| WILMS.TUMOR | ALDH8A1 |
| WILMS.TUMOR | AMDHD1 |
| WILMS.TUMOR | ANGPTL3 |
| WILMS.TUMOR | BACH2 |
| WILMS.TUMOR | CCDC88A |
| WILMS.TUMOR | CDH7 |
| WILMS.TUMOR | CPN2 |
| WILMS.TUMOR | CPXM1 |
| WILMS.TUMOR | CYP17A1 |
| WILMS.TUMOR | CYP27B1 |
| WILMS.TUMOR | CYP4A11 |
| WILMS.TUMOR | CYP4F2 |
| WILMS.TUMOR | CYP8B1 |
| WILMS.TUMOR | DMGDH |
| WILMS.TUMOR | DMRT3 |
| WILMS.TUMOR | DOCK8-AS1 |
| WILMS.TUMOR | DPYS |
| WILMS.TUMOR | EYA1 |
| WILMS.TUMOR | FCAMR |
| WILMS.TUMOR | G6PC |
| WILMS.TUMOR | GBA3 |
| WILMS.TUMOR | GC |
| WILMS.TUMOR | GLYAT |
| WILMS.TUMOR | GLYATL1 |
| WILMS.TUMOR | HOGA1 |
| WILMS.TUMOR | HSPA4L |
| WILMS.TUMOR | IGSF6 |
| WILMS.UMOR | KCNJ10 |
| WILMS.TUMOR | LRRC19 |
| WILMS.TUMOR | LYPD1 |
| WILMS.TUMOR | MEOX1 |
| WILMS.TUMOR | MEX3B |
| WILMS.TUMOR | MIOX |
| WILMS.TUMOR | MN1 |
| WILMS.TUMOR | NAT8 |
| WILMS.TUMOR | PLG |
| WILMS.TUMOR | PLPPR1 |
| WILMS.TUMOR | SIX1 |
| WILMS.TUMOR | SIX2 |
| WILMS.TUMOR | SLC13A1 |
| WILMS.TUMOR | SLC13A3 |
| WILMS.TUMOR | SLC17A1 |
| WILMS.TUMOR | SLC17A3 |
| WILMS.TUMOR | SLC22A11 |
| WILMS.TUMOR | SLC22A12 |
| WILMS.TUMOR | SLC22A2 |
| WILMS.TUMOR | SLC23A3 |
| WILMS.TUMOR | SLC2A2 |
| WILMS.TUMOR | SLC5A12 |
| WILMS.TUMOR | SLC6A12 |
| WILMS.TUMOR | SLC7A13 |
| WILMS.TUMOR | SLC7A9 |
| WILMS.TUMOR | ST8SIA4 |
| WILMS.TUMOR | TENM4 |
| WILMS.TUMOR | TINAG |
| WILMS.TUMOR | UGT1A6 |

### Example 4. Al-Based Primary Tumor Site Determination Model and Verification

As a classification model, Bossitng Decision Tree, ANN, DNN, Regression, etc. were used to train data, and the result value for each algorithm was measured using a verification data set.

The number of data used for training by tumor type and AUROC results by classification algorithm are shown in the tables below.

TABLE 4

| | | | VALIDATION SET (30%) AUROC RESULT | | | | | |
|---|---|---|---|---|---|---|---|---|
| Type of | | | LOGISTIC | | RANDOM | | Gradient | |
| classfication | CD_CANCER | NUMBER | REGRESSION | SVM | FOREST | AdaBoost | Boosting | DNN |
| Binary | ACC | 123 | 0.9709 | 0.5000 | 0.9553 | 0.9456 | 0.9021 | 0.9714 |
| Classfication | ADC | 1007 | 0.9497 | 0.7543 | 0.9703 | 0.9714 | 0.9562 | 0.9799 |
| | ATC | 56 | 0.8279 | 0.5000 | 0.8481 | 0.8928 | 0.9015 | 0.9372 |
| | BCC | 17 | 0.7627 | 0.5882 | 0.5882 | 0.9412 | 0.9411 | 0.9412 |
| | BDC | 198 | 0.9353 | 0.8914 | 0.7727 | 0.9722 | 0.8657 | 0.9924 |
| | BLC | 310 | 0.9915 | 0.5000 | 0.9935 | 0.9984 | 0.9726 | 0.9984 |
| | BREAST. | 5544 | 0.9976 | 0.9372 | 0.9973 | 0.9998 | 0.9999 | 0.9990 |
| | CANCER | | | | | | | |
| | CERVICAL. | 160 | 0.9582 | 0.7688 | 0.8938 | 0.9906 | 0.8901 | 0.9843 |
| | CANCER | | | | | | | |
| | COLON. | 2871 | 0.9984 | 0.9257 | 0.9965 | 0.9998 | 0.9991 | 0.9985 |
| | CANCER | | | | | | | |
| | EAC | 35 | 0.8411 | 0.5000 | 0.9286 | 0.9857 | 0.8284 | 0.9857 |
| | ESCC | 44 | 0.8509 | 0.5000 | 0.6250 | 0.9545 | 0.7153 | 0.9545 |
| | GMB | 956 | 0.8979 | 0.8108 | 0.8587 | 0.8948 | 0.8857 | 0.9313 |
| | GIST | 71 | 0.9924 | 0.5000 | 0.9858 | 0.9858 | 0.9153 | 0.9999 |
| | HBL | 44 | 0.9653 | 0.5000 | 0.8750 | 0.9545 | 0.8974 | 0.9772 |
| | HCC | 413 | 0.9875 | 0.6441 | 0.9587 | 0.9939 | 0.9511 | 0.9891 |
| | HGBT | 587 | 0.8624 | 0.7065 | 0.8019 | 0.8162 | 0.5141 | 0.8766 |
| | HL | 130 | 0.9958 | 0.6115 | 0.9692 | 0.9807 | 0.9729 | 0.9961 |
| | LCC | 56 | 0.5606 | 0.5000 | 0.5179 | 0.5088 | 0.5616 | 0.5709 |
| | LGBT | 976 | 0.8929 | 0.7193 | 0.8680 | 0.8929 | 0.8824 | 0.9313 |
| | MCC | 19 | 0.8667 | 0.5000 | 0.8158 | 0.9211 | 0.8420 | 0.9474 |
| | MM | 41 | 0.9994 | 0.5488 | 0.9146 | 0.9756 | 0.8778 | 0.9878 |
| | NHL | 103 | 0.9751 | 0.5485 | 0.9369 | 0.9854 | 0.8831 | 0.9854 |
| | NPC | 46 | 0.9670 | 0.5000 | 0.9130 | 0.9674 | 0.9782 | 0.9783 |
| | OVARIAN. | 1143 | 0.9962 | 0.9234 | 0.9899 | 0.9996 | 0.9921 | 0.9996 |
| | CANCER | | | | | | | |
| | PANCREATIC. | 207 | 0.9751 | 0.9034 | 0.9034 | 0.9903 | 0.9709 | 0.9807 |
| | CANCER | | | | | | | |
| | PNET | 86 | 0.6209 | 0.5057 | 0.4999 | 0.5985 | 0.5853 | 0.7198 |
| | PPC | 40 | 0.9746 | 0.5000 | 1.0000 | 0.9875 | 1.0000 | 1.0000 |
| | PPGLs | 199 | 0.9914 | 0.5000 | 0.9749 | 0.9925 | 0.9824 | 0.9949 |
| | PROSTATE. | 247 | 0.8003 | 0.9251 | 0.9130 | 0.9960 | 0.9554 | 0.9919 |
| | CANCER | | | | | | | |
| | RCC | 348 | 0.9863 | 0.8405 | 0.9799 | 0.9899 | 0.9637 | 0.9927 |
| | RECTAL. | 198 | 0.9694 | 0.5000 | 0.9773 | 0.9924 | 0.9646 | 0.9949 |
| | CANCER | | | | | | | |
| | SARCOMA | 830 | 0.9952 | 0.6789 | 0.9976 | 0.9988 | 0.9968 | 0.9988 |
| | SCC | 356 | 0.9206 | 0.5969 | 0.9181 | 0.9221 | 0.8964 | 0.9373 |
| | SCLC | 44 | 0.7946 | 0.5000 | 0.7159 | 0.8295 | 0.7607 | 0.8749 |
| | SKIN. | 249 | 0.9833 | 0.5141 | 0.9497 | 0.9699 | 0.9333 | 0.9880 |
| | MELANOMA | | | | | | | |
| | SOMACH. | 920 | 0.9915 | 0.7609 | 0.9815 | 0.9956 | 0.9842 | 0.9933 |
| | CANCER | | | | | | | |
| | UTERINE. | 162 | 0.9993 | 0.7099 | 0.9506 | 0.9907 | 0.9009 | 0.9907 |
| | CANCER | | | | | | | |
| | UVEAL. | 29 | 0.9985 | 0.5000 | 0.9655 | 0.9655 | 0.9483 | 1.0000 |
| | MELANOMA | | | | | | | |
| | WILMS. | 65 | 0.9533 | 0.5000 | 0.8769 | 0.9308 | 0.8228 | 0.9384 |
| | TUMOR | | | | | | | |
| | cScc | 45 | 0.9437 | 0.5111 | 0.8444 | 0.9332 | 0.8774 | 0.9332 |
| | non.ATC | 242 | 0.9745 | 0.8264 | 0.9441 | 0.9730 | 0.9313 | 0.9751 |
| | non.NPC | 576 | 0.9792 | 0.9219 | 0.9800 | 0.9991 | 0.9948 | 0.9947 |
| Multiple | 42 CLASS | | | | 0.9404 | 0.7165 | 0.9104 | 0.7581 |
| Classfication | | | | | | | | |

TABLE 5

| | Logistic | | RANDOM | | Gradient | |
|---|---|---|---|---|---|---|
| Classification | Regression | SVM | FOREST | AdaBoost | Boosting | DNN |
| Carcinoma mean | 92.85% | 66.46% | 88.92% | 94.32% | 87.85% | 95.74% |
| Maximum accuracy | 99.94% | 93.72% | 100.00% | 99.98% | 99.99% | 100.00% |
| Minimum accuracy | 56.06% | 50.00% | 49.99% | 50.88% | 0.00% | 57.09% |
| Carcinoma rates with | 61.90% | 0.00% | 42.86% | 71.43% | 38.10% | 71.43% |
| 95% or higher | | | | | | |
| accuracy | | | | | | |
| Carcinoma rates with | 73.81% | 14.29% | 64.29% | 83.33% | 57.14% | 90.48% |
| 90% or higher | | | | | | |
| accuracy | | | | | | |

TABLE 6

| | Logistic | | RANDOM | | Gradient | |
|---|---|---|---|---|---|---|
| Classification | Regression | SVM | FOREST | AdaBoost | Boosting | DNN |
| First Candidate | 98.10% | 94.84% | 99.74% | 97.87% | 99.05% | 99.31% |
| Accuracy | | | | | | |
| First or Second | 99.36% | 97.02% | 100.00% | 99.69% | 99.82% | 99.98% |
| Candidate | | | | | | |
| Accuracy | | | | | | |

TABLE 7

| | Logistic | | | | Random | | | | Gradient | | | |
|---|---|---|---|---|---|---|---|---|---|---|---|---|
| | Regression | | SVM | | Forest | | AdaBoost | | Boosting | | DNN | |
| | Sensi- | Pecu- | Sensi- | Pecu- | Sensi- | Pecu- | Sensi- | Pecu- | Sensi- | Pecu- | Sensi- | Pecu- |
| Classification | tivity | liarity | tivity | liarity | tivity | liarity | tivity | liarity | tivity | liarity | tivity | liarity |
| ACC | 99.2% | 100.0% | 100.0% | 100.0% | 100.0% | 100.0% | 100.0% | 100.0% | 100..0% | 100..0% | 100..0% | 100..0% |
| PPGLs | 99.0% | 100.0% | 0.0 | — | 100.0% | 100.0% | 100.0% | 100.0% | 100..0% | 100..0% | 100..0% | 100..0% |
| BDC | 99.5% | 99.5% | 100.0% | 97.1% | 100.0% | 100.0% | 100.0% | 100.0% | 100..0% | 100..0% | 100..0% | 100..0% |
| BLC | 99.7% | 99.4% | 1.3% | 0.7% | 100.0% | 100.0% | 100.0% | 100.0% | 100..0% | 100..0% | 100..0% | 100..0% |
| GBM | 92.1% | 86.2% | 97.5% | 95.2% | 99.1% | 97.8% | 92.9% | 84.5% | 96.3% | 91.1% | 99.9% | 93.2% |
| HGBT | 81.8% | 91.9% | 92.3% | 96.6% | 97.1% | 97.9% | 79.3% | 89.1% | 88.6% | 96.1% | 87.4% | 99.5% |
| LGBT | 94.2% | 91.5% | 97.0% | 95.3% | 98.8% | 98.7% | 90.5% | 87.9% | 95.9% | 94.4% | 97.8% | 94.5% |
| PNET | 69.8% | 90.9% | 93.0% | 100.0% | 93.0% | 100.0% | 65.1% | 100.0% | 90.7% | 98.7% | 94.2% | 94.2% |
| BREAST. | 99.7% | 99.7% | 100.0% | 99.9% | 100.0% | 100.0% | 100.0% | 100.0% | 100.0% | 100.0% | 100.0% | 100.0% |
| CANCER | | | | | | | | | | | | |
| COLON. | 100.0% | 100.0% | 100.0% | 100.0% | 100.0% | 100.0% | 100.0% | 100.0% | 100.0% | 100.0% | 100.0% | 100.0% |
| CANCER | | | | | | | | | | | | |
| EAC | 100.0% | 100.0% | 0.0 | — | 100.0% | 100.0% | 100.0% | 100.0% | 100.0% | 100.0% | 100.0% | 100.0% |
| ESCC | 100.0% | 100.0% | 100.0% | 100.0% | 100.0% | 100.0% | 100.0% | 100.0% | 100.0% | 95.7% | 100.0% | 100.0% |
| GIST | 100.0% | 100.0% | 0.0 | — | 100.0% | 100.0% | 100.0% | 100.0% | 100.0% | 100.0% | 100.0% | 100.0% |
| STOMACH. | 99.3% | 98.5% | 100.0% | 100.0% | 100.0% | 100.0% | 100.0% | 100.0% | 100.0% | 100.0% | 100.0% | 100.0% |
| CANCER | | | | | | | | | | | | |
| NPC | 100.0% | 100.0% | 100.0% | 100.0% | 100.0% | 100.0% | 100.0% | 100.0% | 100.0% | 100.0% | 100.0% | 100.0% |
| non.NPC | 98..6% | 96.1% | 100.0% | 99.8% | 100.0% | 100.0% | 100.0% | 100.0% | 100.0% | 100.0% | 100.0% | 100.0% |
| HCC | 99.8% | 100.0% | 100.0% | 99.3% | 100.0% | 100.0% | 100.0% | 100.0% | 100.0% | 100.0% | 100.0% | 100.0% |
| HBL | 100.0% | 100.0% | 4.5% | 100.0% | 100.0% | 100.0% | 100.0% | 100.0% | 100.0% | 100.0% | 100.0% | 100.0% |
| OVARIAN. | 99.7% | 100.0% | 100.0% | 99.9% | 100.0% | 100.0% | 100.0% | 100.0% | 100.0% | 99.9% | 100.0% | 100.0% |
| CANCER | | | | | | | | | | | | |
| PANCREATIC. | 98.1% | 100.0% | 100.0% | 95.4% | 100.0% | 100.0% | 100.0% | 100.0% | 100.0% | 100.0% | 100.0% | 100.0% |
| CANCER | | | | | | | | | | | | |
| PROSTATE. | 100.0% | 100.0% | 100.0% | 98.8% | 100.0% | 100.0% | 100.0% | 100.0% | 100.0% | 100.0% | 100.0% | 100.0% |
| CANCER | | | | | | | | | | | | |
| RECTAL. | 100.0% | 100.0% | 99.5% | 100.0% | 100.0% | 100.0% | 100.0% | 100.0% | 100.0% | 100.0% | 100.0% | 100.0% |
| CANCER | | | | | | | | | | | | |
| PPC | 100.0% | 100.0% | 0.0 | — | 100.0% | 100.0% | 100.0% | 100.0% | 100.0% | 100.0% | 100.0% | 100.0% |
| RCC | 99.4% | 100.0% | 99.4% | 100.0% | 100.0% | 100.0% | 100.0% | 100.0% | 100.0% | 100.0% | 100.0% | 100.0% |
| WILMS. | 100.0% | 100.0% | 98.5% | 100.0% | 100.0% | 100.0% | 100.0% | 100.0% | 100.0% | 100.0% | 100.0% | 100.0% |
| TUMOR | | | | | | | | | | | | |
| SARCOMA | 100.0% | 100.0% | 99.9% | 100.0% | 100.0% | 100.0% | 100.0% | 100.0% | 100.0% | 100.0% | 100.0% | 100.0% |
| SKIN. | 99.6% | 99.6% | 99.6% | 99.2% | 100.0% | 100.0% | 100.0% | 100.0% | 100.0% | 100.0% | 100.0% | 100.0% |
| MELANOMA | | | | | | | | | | | | |
| cSCC | 100.0% | 100.0% | 100.0% | 100.0% | 100.0% | 100.0% | 100.0% | 100.0% | 100.0% | 100.0% | 97.8% | 100.0% |
| BCC | 100.0% | 100.0% | 100.0% | 100.0% | 100.0% | 100.0% | 100.0% | 100.0% | 100.0% | 100.0% | 100.0% | 100.0% |
| MCC | 100.0% | 100.0% | 0.0 | — | 100.0% | 100.0% | 100.0% | 100.0% | 100.0% | 100.0% | 100.0% | 100.0% |
| ATC | 94.6% | 100.0% | 0.0 | — | 100.0% | 100.0% | 100.0% | 100.0% | 100.0% | 100.0% | 100.0% | 100.0% |
| non.ATC | 98.3% | 98.8% | 98.8% | 99.2% | 100.0% | 100.0% | 100.0% | 100.0% | 100.0% | 100.0% | 100.0% | 100.0% |
| UTERINE. | 100.0% | 100.0% | 100.0% | 100.0% | 100.0% | 100.0% | 100.0% | 100.0% | 100.0% | 100.0% | 100.0% | 100.0% |
| CANCER | | | | | | | | | | | | |
| CERVICAL. | 99.4% | 100.0% | 99.4% | 100.0% | 100.0% | 100.0% | 100.0% | 100.0% | 100.0% | 100.0% | 100.0% | 100.0% |
| CANCER | | | | | | | | | | | | |
| UVEAL. | 100.0% | 100.0% | 0.0 | — | 100.0% | 100.0% | 100.0% | 100.0% | 100.0% | 100.0% | 100.0% | 96.7% |
| MELANOMA | | | | | | | | | | | | |
| HL | 99.2% | 97.7% | 100.0% | 100.0% | 100.0% | 100.0% | 100.0% | 100.0% | 100.0% | 100.0% | 100.0% | 100.0% |
| NHL | 99.0% | 86.4% | 100.0% | 100.0% | 100.0% | 100.0% | 100.0% | 100.0% | 100.0% | 100.0% | 100.0% | 100.0% |
| MM | 100.0% | 100.0% | 100.0% | 100.0% | 100.0% | 100.0% | 100.0% | 100.0% | 100.0% | 100.0% | 100.0% | 100.0% |
| ADC | 99.7% | 99.1% | 100.0% | 97.8% | 100.0% | 100.0% | 98.6% | 96.0% | 99.9% | 99.2% | 99.9% | 99.8% |
| SCC | 100.0% | 100.0% | 99.4% | 99.4% | 100.0% | 100.0% | 93.5% | 94.9% | 98.3% | 99.7% | 100.0% | 99.7% |
| LCC | 89.9% | 100.0% | 0.0 | — | 100.0% | 100.0% | 58.9% | 97.1% | 96.4% | 100.0% | 96.4% | 100.0% |
| SCLC | 100.0% | 100.0% | 0.0 | — | 100.0% | 100.0% | 100.0% | 100.0% | 100.0% | 100.0% | 100.0% | 100.0% |

What is claimed is:

1. A method for determining a primary tumor site, the method comprising:
acquiring gene expression data of a biological sample including tumor cells of which a primary site is not specified; and
classifying the primary site of the biological sample into one of a plurality of tumor types by comparing the gene expression data of the biological sample with specific gene expression data for each of the plurality of tumor types using a classification algorithm.
